(19) United States
(12) Patent Application Publication
(10) Pub. No.: US 2024/0299204 A1
(43) Pub. Date: Sep. 12, 2024

(54) VACUUM-ASSISTED EVACUATION OF MALE EXTERNAL CATHETERS

(71) Applicant: Stephen L. Rawlins, Lehi, UT (US)

(72) Inventor: Stephen L. Rawlins, Lehi, UT (US)

(21) Appl. No.: 18/235,027

(22) Filed: Aug. 17, 2023

Related U.S. Application Data
(63) Continuation-in-part of application No. 18/117,761, filed on Mar. 6, 2023.

(51) Int. Cl. A61F 5/453; A61F 5/44

(52) U.S. Cl. CPC A61F 5/453; A61F 5/4405

(57) ABSTRACT

An apparatus for collecting urine from a user includes a sheath portion having a first end and a second end, wherein the first end is configured to make a seal with a penis of a user. An outlet is positioned at the second end and is configured to enable urine to exit the sheath portion. A channel is operably coupled to the outlet to convey urine away from the sheath portion. A throttle valve is coupled to the channel at or near the outlet to enable air to enter the channel in a regulated manner. In certain embodiments, the channel is coupled to a collection chamber which is in turn coupled to a vacuum device. The vacuum device may be configured to pull air bubbles into the channel to assist in conveying the urine into the collection chamber. A corresponding method is also disclosed.

### BACKGROUND

### Field of the Invention

This invention relates to catheters, and more particularly to male external urinary catheters that can be evacuated with the assistance of a vacuum device.

### Background of the Invention

Urinary catheters are used extensively in the medical field to empty a patient's bladder into a bag or other container. Such catheters may be used for health issues such as urinary incontinence, urinary retention, surgery, immobility, or the like. Urinary catheters come in various different types including indwelling catheters, external catheters, intermittent catheters, and the like. They may also be specifically designed for male or female use. However, whatever type a catheter may be, they are almost always designed for use in professional or commercial settings. They are also typically designed to be disposable and thus are intended to be replaced on a regular basis.

What is missing in the marketplace is a urinary catheter that is geared toward a more casual user in a non-medical setting. For example, a casual user could use a catheter in his or her home to urinate and thereby avoid having to get up in the middle of the night and interrupt the user's rest or sleep. This may be particularly useful with older users or users that have to urinate frequently or that have impaired mobility. Other potential users may include campers, truckers, or workers that may not have convenient or frequent access to bathrooms. Ideally such a catheter would be inexpensive, easy to clean, and reusable to cut down on waste. Further needed are techniques to effectively evacuate the catheter of urine that go beyond simply relying on gravity.

### SUMMARY

The invention has been developed in response to the present state of the art and, in particular, in response to the problems and needs in the art that have not yet been fully solved by currently available apparatus and methods. Accordingly, apparatus and methods in accordance with the invention have been developed to collect urine from a user. The features and advantages of the invention will become more fully apparent from the following description and appended claims, or may be learned by practice of the invention as set forth hereinafter.

Consistent with the foregoing, an apparatus for collecting urine from a user includes a sheath portion having a first end and a second end, wherein the first end is configured to make a seal with a penis of a user. An outlet is positioned at the second end and is configured to enable urine to exit the sheath portion. A channel is operably coupled to the outlet to convey urine away from the sheath portion. A throttle valve is coupled to the channel at or near the outlet to enable air to enter the channel in a regulated manner. In certain embodiments, the channel is coupled to a collection chamber which is in turn coupled to a vacuum device. The vacuum device may be configured to pull air bubbles into the channel through the throttle valve to assist in conveying the urine through the channel and into the collection chamber.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings, in which:

FIG. 1 is a perspective view showing one embodiment of a urinary catheter in accordance with the invention, including a check valve installed near an outlet thereof;

FIG. 2 is a perspective view showing the urinary catheter of FIG. 1, and more particularly showing air bubbles being pulled into the channel through the check valve to assist with conveying urine through the channel;

FIG. 3 is perspective view of a system including the urinary catheter, a collection chamber, and a vacuum device to pull urine into the collection chamber;

FIG. 4 is a perspective view of a more particular embodiment of the system of FIG. 3; and

FIG. 5 is a perspective view showing one embodiment of a urinary catheter in accordance with the invention, including a throttle valve installed near an outlet thereof.

### DETAILED DESCRIPTION

It will be readily understood that the components of the present invention, as generally described and illustrated in the Figures herein, could be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

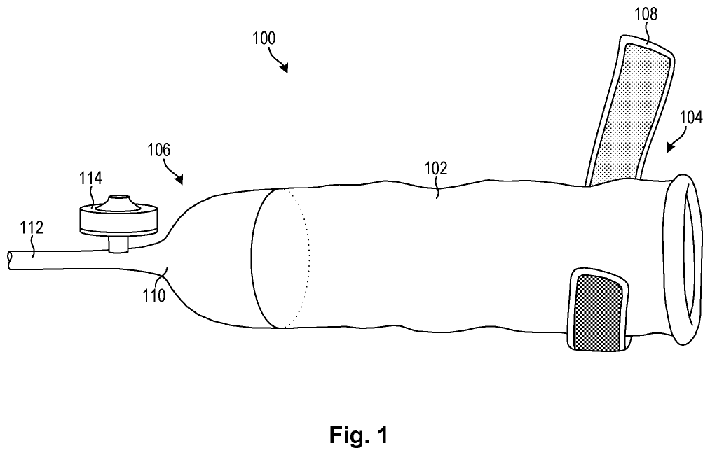

Referring to FIG. 1, as previously mentioned, urinary catheters are used extensively in the medical field to empty a patient's bladder into a bag or other container. Such catheters may be used for health issues such as urinary incontinence, urinary retention, surgery, immobility, or the like. Urinary catheters come in various different types, including indwelling catheters, external catheters, intermittent catheters, and the like. They may also be specifically designed for male or female use. However, whatever type a catheter may be, they are almost always designed for use in professional or commercial settings. They are also typically designed to be disposable and thus are intended to be replaced on a regular basis.

What is missing in the marketplace is a urinary catheter that is geared toward a more casual user for use in a non-medical setting. For example, a casual user could potentially use a urinary catheter in his or her home to urinate and thereby avoid having to get up in the middle of the night and interrupt the user's rest or sleep. This may be particularly useful with older users, users that have to urinate frequently due to a medical or other condition, or users that have impaired mobility. Other potential users may include campers, truckers, or workers that may not have convenient or frequent access to bathrooms. Ideally such a catheter would be inexpensive, easy to clean, and reusable to cut down on waste. Further needed are techniques to effectively evacuate the catheter of urine that go beyond simply relying on gravity.

FIG. 1 is a perspective view showing one embodiment of a urinary catheter 100 in accordance with the invention. The urinary catheter 100 shown in FIG. 1 may also be referred to as a “condom catheter 100”. As shown, the urinary catheter 100 includes a sheath portion 102 having a first end 104 and a second end 106. The first end 104 is configured to make an air and/or liquid tight seal with a penis of a user. The seal may be generated by a fit of the sheath portion 102 around the penis of a user and/or additional mechanisms, such as the illustrated strap 108 with hook and loop fasteners, that may be tightened around the penis of a user in order to enhance the seal of the sheath portion 102 around the penis.

The urinary catheter 100 may be referred to as a “condom catheter” since it may fit on the penis like a condom. The urinary catheter 100 may also be referred to as an external urinary catheter or a penile sheath catheter. The urinary catheter 100 may be made of any suitable material such as latex or silicon. It may also be provided in different sizes. Although the urinary catheter 100 is shown with a strap 108, the urinary catheter 100 may also be attached to a user with an adhesive, tape, or the like.

The second end 106 of the sheath portion 102 includes an outlet 110 that enables urine to exit the sheath portion 102. A urine transport channel 112 (e.g., a flexible tube) may be operably coupled to this outlet 110 to enable the urine to be conveyed away from the urinary catheter 100. In some cases, the urine may flow away under the influence of gravity. This may be possible in applications where the user is standing and any collection chamber is below the level of the urinary catheter 100. Unfortunately, such gravity-based systems may, in certain cases, leave the penis bathed in urine. To prevent this from occurring, the urine may also be conveyed away from the urinary catheter 100 using suction, such as that generated by a vacuum device. This may be more useful in applications where the user is seated or laying down or the urine collection chamber is at or above the level of the urinary catheter 100. This may also more effectively evacuate urine from the urinary catheter 100 to leave a user more comfortable between urination events.

In order to facilitate conveyance of urine away from the urinary catheter 100, a check valve 114 may be coupled to the urine transport channel 112 at or near the outlet 110. This may enable air to enter the urine transport channel 112 and urinary catheter 100 to replace urine that is drawn out of the urine transport channel 112 by the vacuum device. The check valve 114 may also prevent air and/or liquids from exiting the urine transport channel 112 through the check valve 114. Ideally, the check valve 114 is placed as close to the outlet 110 as possible. For example, in certain embodiments, the check valve 114 is placed within one inch of the outlet 110. In other embodiments, the check valve 114 is placed within two inches of the outlet 110.

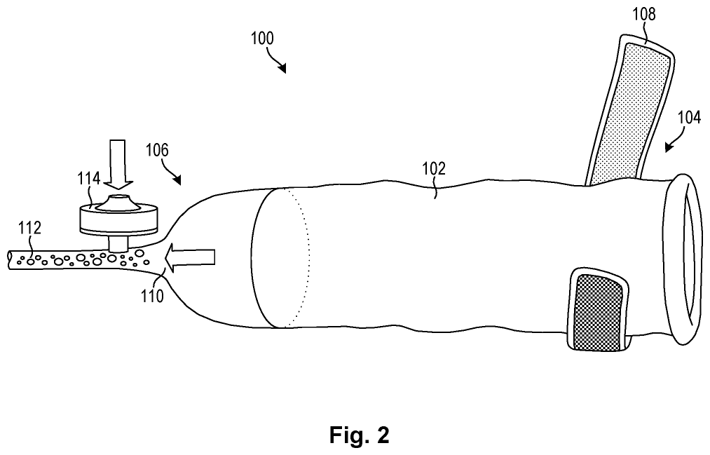

As shown in FIG. 2, as urine is drawn away from the urinary catheter 100 through the urine transport channel 112 using a vacuum, air may enter the urine transport channel 112 through the check valve 114. Because the sheath portion 102 essentially forms a sealed container with the penis, the air may replace the urine that is drawn out of the urinary catheter 100. In addition, air bubbles that enter through the check valve 114 may travel up the urine transport channel 112 with the urine to assist in conveying the urine to a collection chamber.

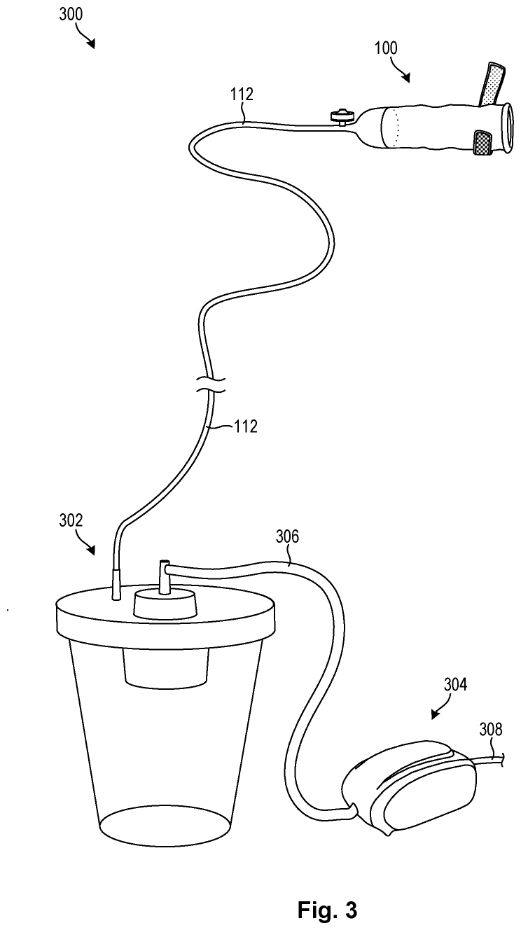

Referring to FIG. 3, one embodiment of a system 300 for collecting urine from a user is illustrated. As shown, the system 300 includes the urinary catheter 100 shown in FIGS. 1 and 2, a collection chamber 302, and a vacuum device 304 used to pull urine into the collection chamber 302. In the illustrated embodiment, the vacuum device 304 (powered through a cord 308) generates a vacuum within the collection chamber 302 by way of a channel 306 (e.g., a flexible tube). This may in turn generate suction in the urine transport channel 112 to pull urine from the urinary catheter 100 to the collection chamber 302. The urine transport channel 112 may only need to be connected to the collection chamber 302 while the user is urinating. During other times, the urine transport channel 112 may be disconnected from the collection chamber 302 and closed with a cap (e.g., a rubber cap) to allow a user of the urinary catheter 100 to move about uninhibited. In the illustrated embodiment, the vacuum device 304 is separate from the collection chamber 302, although the vacuum device 304 and the collection chamber 302 may also be incorporated into a single device in other embodiments. For example, the vacuum device 304 may be housed within a compartment underneath the collection chamber 302.

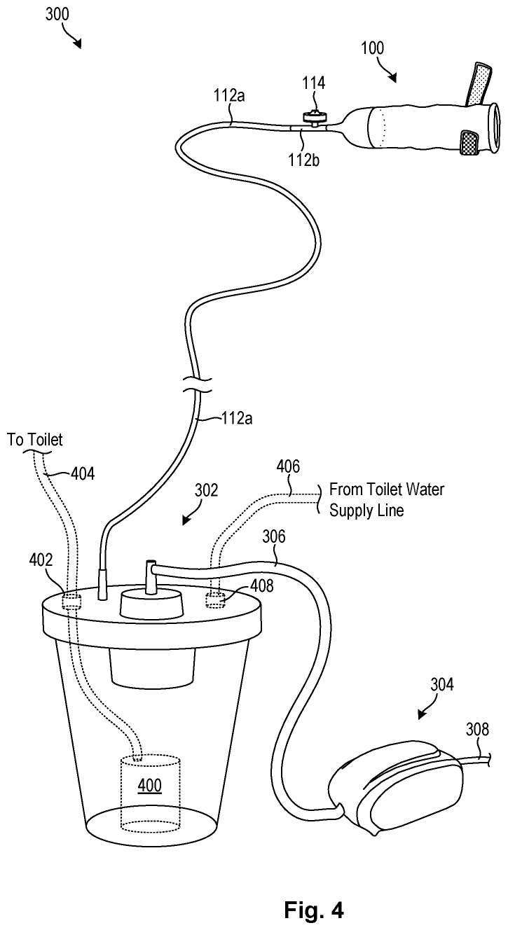

Referring to FIG. 4, in one particular embodiment in accordance with the invention, a system 300 in accordance with the invention includes the following: (1) an air tight collection chamber 302 capable of handling low vacuum and of sufficient size to incorporate a small submersible sump pump; (2) a urine transfer tube 112a, such as a ¼ inch inside diameter by ⅜ inch outside diameter vinyl tube, about six feet long connected to the collection chamber 302 on one end a ⅜ inch outside diameter coupler on the other end to connect to the catheter tube 112b; (3) a catheter tube 112b consisting of a short length of ⅜ inch outside diameter tube with a small plastic barbed one-way air entry valve 114 inserted near the end that the external urinary catheter 100 is connected to, with the urine transfer tube 112a connected to the other end; (4) a small vacuum device 304 like those typically used for filtration in laboratories connected by a tube 306 through a top of the collection chamber 302; (5) a small submersible sump pump 400 that automatically turns on when liquid rises above a given level and turns off when the liquid level reaches a bottom of the collection chamber 302 or below a certain level, where an electrical connection (not shown) to the pump 400 is fed through an air tight bulkhead fitting through a wall of the collection chamber 302, a bulkhead connector 402 for fluids is installed through a wall of the collection chamber 302 to handle pump effluent, and effluent is transferred through a tube 404 to a toilet or other disposal area; and (6) a needle valve (not shown) connected to a tee in a toilet water supply line and to a small tube 406 to the collection chamber 302, where the tube 406 is connected to a float valve 408 at a top of the collection chamber 302.

In operation, the sump pump 400 may be kept in an active state to remove liquid from the collection chamber 302 when it reaches a high limit and turn off when the collection chamber 302 is empty or reaches a lower limit. To flush the collection chamber 302, water may be supplied slowly through the tube 406 into the collection chamber 302 from a toilet water supply line. The needle valve may be adjusted to supply sufficient water to flush the collection chamber 302 during the day. For example, the needle valve may be set to supply about one pint per hour. This provides about three gallons of water during a twenty-four hour period. The collection chamber 302 may empty while the sump pump 400 is running and fill when it shuts off. The number of flush cycles that occur is controlled by the amount of water that is permitted to flow into the collection chamber 302.

Water may enter the top of the collection chamber 302 through a float valve 408. This float valve 408 may act as a safety feature. If the sump pump were to fail while water is entering the collection chamber 302, the collection chamber 302 could potentially overfill. The float valve 408 may establish a maximum height that water can rise in the collection chamber 302. Normally, a low flow rate in the water supply line 406 will cause the pressure in the line 406 to be very low. However, if the sump pump 400 fails and the float valve 408 closes, pressure in the tube 406 may rise to that in the toilet supply line, which is typically sixty PSI or greater. Therefore, water supply tubing 406 may be selected that is able to withstand the toilet supply line pressure. The pressure in the tubing 404 from the sump pump 400 to the toilet may always be low, so low-pressure tubing may be sufficient.

A manual alternative to providing a continuous water supply to flush the collection chamber 302 is to simply turn on the vacuum device 304 to enable water to be drawn into the collection chamber 302 through the urine transport channel 112a from a container and allowing the sump pump 400 to dispose of the water. This may also provide a convenient way to sanitize the collection chamber 302 and urine transport channel 112a by enabling a dilute detergent and bleach solution to be drawn into the collection chamber 302 and thereafter disposed of.

As convenient as it may be to have a system 300 with automated urine disposal, one downside of such a system 300 is that it may only be feasible as a fixed system. A much more versatile system 300 may include a portable collection chamber 302 that is manually emptied but provides additional applications beyond just bedroom sleeping, such as with a sleeping bag on a tent floor or midway between rest stops on a long desert highway.

With the urinary catheter 100 dry and comfortable to wear, it may be disconnected from the collection chamber 302 with only a rubber capped catheter tube 112b positioned at a user's belt line. This gives the user complete freedom except when the need to use the system 300 arises. It may then be reconnected to the urine transport channel 112a and the vacuum device 304 may be activated.

Although only a male external urinary catheter 100 is shown with the system 300, it may work equally well with female external urinary catheters, except in this application the one-way check valve 114 may be omitted since air may enter the urine transport channel 112a through the catheter itself.

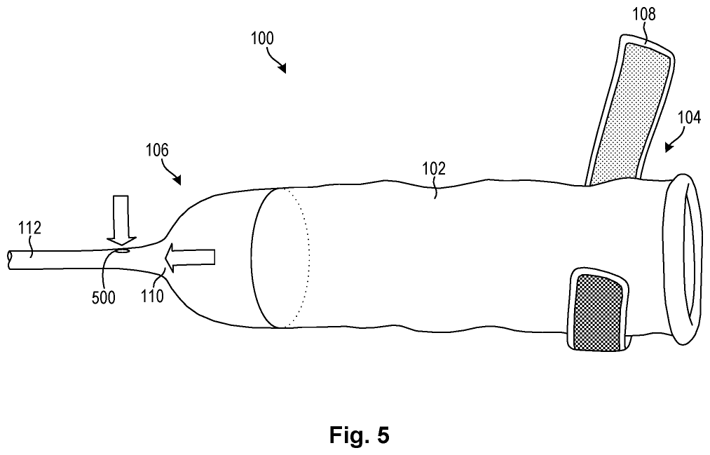

Referring to FIG. 5, in certain embodiments in accordance with the invention, the check valve 114 shown in FIGS. 1 through 4 may be replaced with a throttle valve 500. In certain embodiments, the throttle valve 500 is implemented as a small hole or orifice that is located in the urine transport channel 112 at or near the outlet 110 of the urinary catheter 100. This throttle valve 500 may enable air to enter the urine transport channel 112 in a regulated manner to convey urine up the urine transport channel 112. The throttle valve 500 may be finely adjusted to permit sufficient air flow to enter the urine transport channel 112 without significantly reducing the vacuum therein. In certain embodiments, the adjustment may be made by altering the size (e.g., diameter) of any hole or orifice in the throttle valve 500. Incidentally, these same types of features and adjustments may be made for the check valve 114 previously discussed.

Experience gained through tests of the system 300 has shown that the urinary catheter 100 is ideally always under vacuum while a user is urinating. Without the vacuum, pressure within the urinary catheter 100 caused by urination may cause the catheter 100 to become dislodged and/or unsealed from the user and thereby cause urine to leak therefrom. In certain embodiments, providing a consistent and constant vacuum while a user urinates reduces or eliminates the need for a one-way check valve 114. Even though liquid may theoretically flow in both directions through the throttle valve 500, under vacuum, air will typically only flow into the urine transport channel 112 through the throttle valve 500 without allowing liquid to escape.

Although leaking has not been found to be an issue, in certain embodiments, a cap or cover may be used to cover the throttle valve 500 when not in use or when a vacuum is not present to prevent the possibility of any urine leaking from the urine transport channel 112. Similarly, in certain embodiments, the throttle valve 500 itself may be adjustable to adjust the rate at which air is allowed to enter the urine transport channel 112. This may be used to compensate for different levels of vacuum that may be present within the urine transport channel 112. For example, in certain embodiments, a screw adjustment may be used to adjust the rate at which air flows through the throttle valve 500 and could also be used to completely turn off air flow though the throttle valve 500 when the system 300 is not in use to prevent leaking.

In the above disclosure, reference has been made to the accompanying drawings which is shown by way of illustration specific implementations in which the disclosure may be practiced. It is understood that other implementations may be utilized and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

While various embodiments of the present disclosure have been described above, it should be understood that they have been presented by way of example only and not limitation. It will be apparent to persons skilled in the relevant art that various changes in form and detail can be made therein without departing from the spirit and scope of the disclosure. Thus, the breadth and scope of the present disclosure should not be limited by any of the above-described exemplary embodiments, but should be defined only in accordance with the following claims and their equivalents. The foregoing description has been presented for the purposes of illustration and description. It is not intended to be exhaustive or to limit the disclosure to the precise form disclosed. Many modifications and variations are possible in light of the above teachings. Further, it should be noted that any or all of the aforementioned alternate implementations may be used in any combination desired to form additional hybrid implementations of the disclosure.

1. An apparatus for collecting urine from a user, the apparatus comprising:
a sheath portion comprising a first end and a second end, the first end configured to make a seal with a penis of a user;
an outlet at the second end and configured to enable urine to exit the sheath portion;
a channel operably coupled to the outlet to convey urine away from the sheath portion; and
a throttle valve operably coupled to the channel at or near the outlet to enable air to enter the channel in a regulated manner.

2. The apparatus of claim 1, wherein the channel is operably coupled to a collection chamber.

3. The apparatus of claim 2, wherein the collection chamber is operably coupled to a vacuum device.

4. The apparatus of claim 3, wherein the vacuum device is configured to pull urine from the sheath portion into the collection chamber.

5. The apparatus of claim 4, wherein the vacuum device is configured to pull air bubbles into the channel through the throttle valve.

6. The apparatus of claim 5, wherein the air bubbles assist in conveying the urine through the channel from the sheath portion to the collection chamber.

7. The apparatus of claim 1, wherein the seal is an air-tight seal.

8. The apparatus of claim 1, wherein the throttle valve enables air to enter at least one of the channel and the sheath portion to replace urine therein.

9. The apparatus of claim 1, wherein the throttle valve is within one inch of the outlet.

10. The apparatus of claim 1, wherein the channel is made up of flexible tubing.

11. A method for collecting urine from a user, the method comprising:
gathering urine in a sheath portion comprising a first end and a second end, the first end configured to make a seal with a penis of a user,
enabling, through an outlet at the second end, urine to exit the sheath portion;
conveying the urine away from the sheath portion through a channel operably coupled to the outlet; and
enabling, using a throttle valve at or near the outlet, air to enter the channel in a regulated manner.

12. The method of claim 11, further comprising conveying, through the channel, the urine to a collection chamber.

13. The method of claim 12, wherein the collection chamber is operably coupled to a vacuum device.

14. The method of claim 13, further comprising pulling, using the vacuum device, the urine from the sheath portion into the collection chamber.

15. The method of claim 14, further comprising pulling, using the vacuum device, air bubbles into the channel through the throttle valve.

16. The method of claim 15, further comprising using the air bubbles to assist in conveying the urine through the channel from the sheath portion to the collection chamber.

17. The method of claim 11, wherein the seal is an air-tight seal.

18. The method of claim 11, further comprising enabling, using the throttle valve, air to enter at least one of the channel and the sheath portion to replace urine therein.

19. The method of claim 11, wherein the throttle valve is within one inch of the outlet.

20. The apparatus of claim 11, wherein the channel is made up of flexible tubing.
